(19) United States
(12) Patent Application Publication
(10) Pub. No.: US 2011/0009931 A1
(43) Pub. Date: Jan. 13, 2011

(54) COOLING PACK DEVICE

(76) Inventor: Rong-Tai Hong, Kaohsiung City (TW)

Correspondence Address: BRINKS HOFER GILSON & LIONE, P.O. BOX 10395, CHICAGO, IL, 60610, US

(21) Appl. No.: 12/725,208

(22) Filed: Mar. 16, 2010

(30) Foreign Application Priority Data
Jul. 13, 2009 (TW) 098212719

(51) Int. Cl. A61F 7/02

(52) U.S. Cl. 607/112

(57) ABSTRACT

A cooling pack device is adapted to be applied to an object, and includes a cooling pack unit and a fastening unit. The cooling pack unit includes first and second cooling packs, each of which has a periphery that has an outer peripheral section formed with at least one fastening ring portion, and a connecting peripheral section interconnecting two ends of the outer peripheral section. The cooling pack unit further includes a pack connecting component that interconnects foldably the connecting peripheral sections of the first and second cooling packs. The fastening unit includes at least one fastening member operable for extending through the fastening ring portions of the outer peripheral sections of the first and second cooling packs so as to fasten the cooling pack unit on the object.

### CROSS-REFERENCE TO RELATED APPLICATION

This application claims priority of Taiwanese Application No. 098212719, filed on Jul. 13, 2009.

### BACKGROUND OF THE INVENTION

1. Field of the Invention

The invention relates to a cooling pack device, more particularly to a portable cooling pack device.

2. Description of the Related Art

A cooling pack is generally used for cooling a body part when the body part is injured or feels pain. In use, the cooling pack is generally held by a user to contact directly the body part so as to cool the body part. The conventional cooling pack is inconvenient to use because, the user has to hold the cooling pack on the body part to maintain the contact between the cooling pack and the body part.

Referring to FIG. 1, Taiwanese Patent Publication No. M292369 discloses a conventional cooling pack device that comprises a cooling pack 11, a fastening belt 12 extending from an end of the cooling pack 11, a cooling substance 13 retained removably in the cooling pack 11, and a ring member 14 connected to an opposite end of the cooling pack 11 that is opposite to the fastening belt 12. The fastening belt 12 includes a belt body and a fastening mechanism 121 provided on the belt body. Before use, the cooling substance 13 has to be frozen in a refrigerator. In use, the cooling substance 13 is taken out from the refrigerator and put into the cooling pack 11. The cooling pack 11 is then sealed by use of a closure unit 111 that is formed thereon. Referring further to FIG. 2, a distal end of the fastening belt 12 can be operated to surround the body part, extend through the ring member 14, form a loop around a part of the ring member 14, and be fastened to the belt body through the fastening mechanism 121, such that the conventional cooling pack device can be fastened on the body part without being held by the user.

However, since the cooling substance 13 is not easily bent after being frozen, when the injured or painful body part has a relatively large area and is located at the vicinity of a joint, the conventional cooling pack device is unable to completely contact the body part, thereby resulting in relatively poor flexibility during use.

### SUMMARY OF THE INVENTION

Therefore, the object of the present invention is to provide a foldable cooling pack device that can overcome the aforesaid drawback associated with the prior art.

Accordingly, a cooling pack device of the present invention is adapted to be applied to an object, and comprises a cooling pack unit and a fastening unit. The cooling pack unit includes first and second cooling packs, each of which has a periphery that has an outer peripheral section formed with at least one fastening ring portion, and a connecting peripheral section interconnecting two ends of the outer peripheral section. The cooling pack unit further includes a pack connecting component that interconnects foldably the connecting peripheral sections of the first and second cooling packs. The fastening unit includes at least one fastening member operable for extending through the fastening ring portions of the outer peripheral sections of the first and second cooling packs so as to fasten the cooling pack unit on the object.

### BRIEF DESCRIPTION OF THE DRAWINGS

Other features and advantages of the present invention will become apparent in the following detailed description of the preferred embodiments with reference to the accompanying drawings, of which:

FIG. 1 is a partly exploded perspective view of a conventional cooling pack device disclosed in Taiwanese Patent Publication No. M292369;

FIG. 2 is a schematic view of the conventional cooling pack device disclosed in Taiwanese Patent Publication No. M292369 in a state of use;

FIG. 3 is a schematic view of a first preferred embodiment of a cooling pack device according to the invention;

FIG. 4 is a schematic view of the first preferred embodiment when in a state of use;

FIG. 5 is a schematic view of a second preferred embodiment of the cooling pack device according to the invention;

FIG. 6 is a schematic view of a third preferred embodiment of the cooling pack device according to the invention;

FIG. 7 is a schematic view of the third preferred embodiment when in a state of use;

FIG. 8 is a fragmentary partly exploded view of a fourth preferred embodiment of the cooling pack device according to the invention;

FIG. 9 is a schematic view of a fifth preferred embodiment of the cooling pack device according to the invention; and

FIG. 10 is a schematic view of a sixth preferred embodiment of the cooling pack device according to the invention.

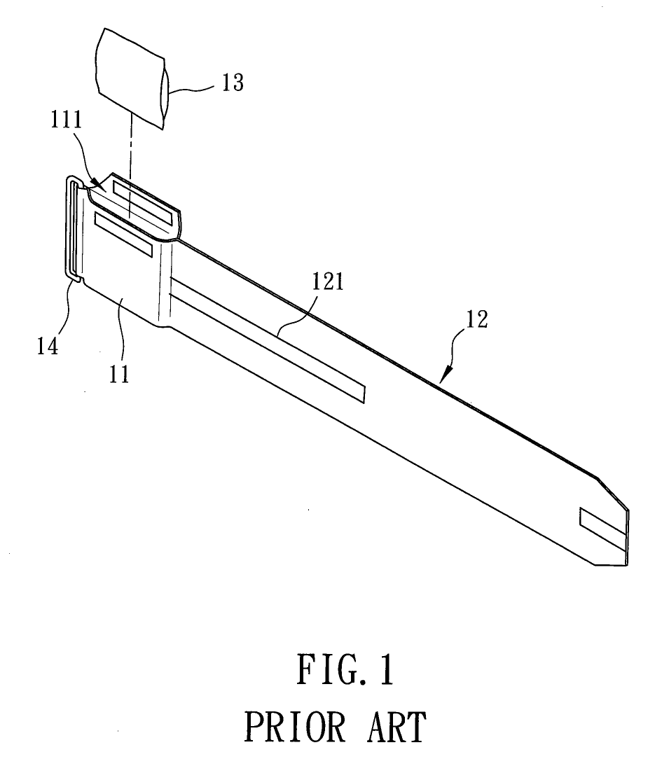

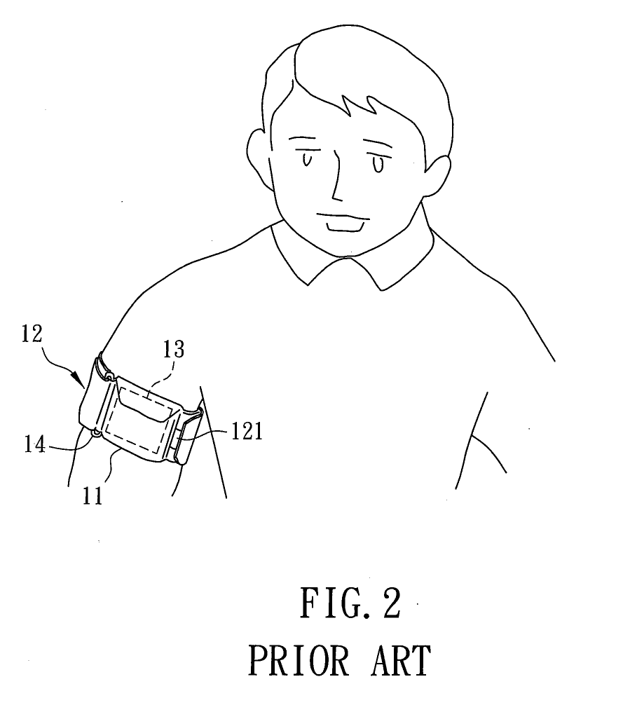

### DETAILED DESCRIPTION OF THE PREFERRED EMBODIMENTS

Before the present invention is described in greater detail, it should be noted that like elements are denoted by the same reference numerals throughout the disclosure.

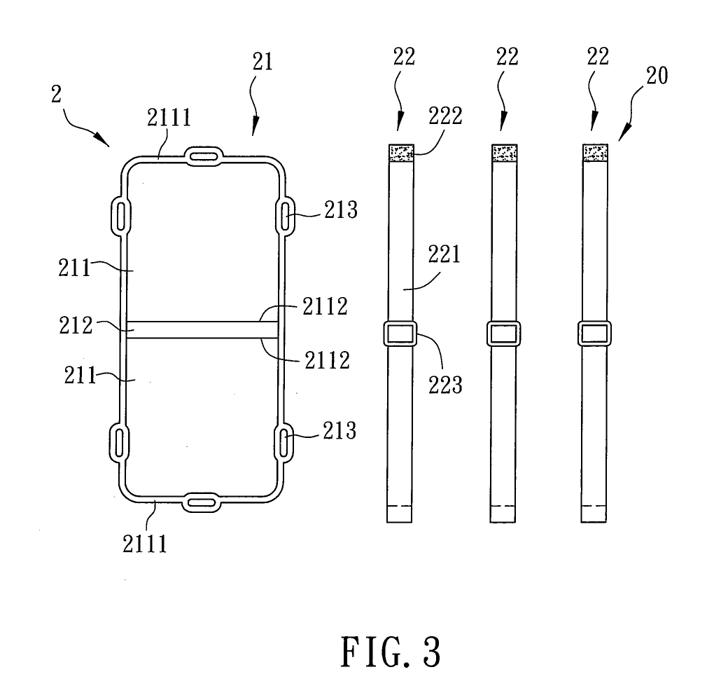

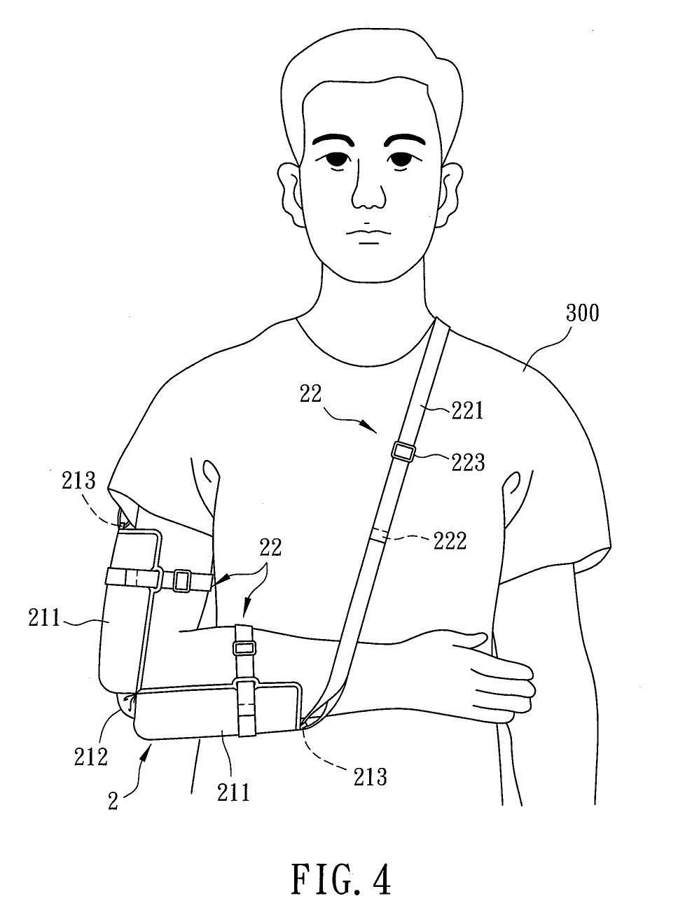

As shown in FIG. 3, the first preferred embodiment of a cooling pack device 2 according to the present invention is adapted to be applied to an object, such as a body part of a user 300 (see FIG. 4). The cooling pack device 2 comprises a first cooling pack unit 21 and a fastening unit 20.

In this embodiment, the first cooling pack unit 21 includes first and second cooling packs 211, each having a periphery that has an outer peripheral section 2111 formed with a plurality of first fastening ring portions 213, and a connecting peripheral section 2112 interconnecting two ends of the outer peripheral section 2111. The first cooling pack unit 21 further includes a first pack connecting component 212 that interconnects foldably the connecting peripheral sections 2112 of the first and second cooling packs 211. Each of the first and second cooling packs 211 is configured as an airtight enclosure and has a cooling substance (not shown) retained non-removably therein.

In this embodiment, the fastening unit 20 includes three fastening members 22. Each of the fastening members 22 includes a connecting band 221 having longitudinally opposite end portions, a fastening mechanism 222 provided on the end portions of the connecting band 221 for connecting releasably the end portions, and an adjusting member 223 provided on the connecting band 221 for adjusting a length of the connecting band 221. The fastening mechanism 222 is configured as a hook-and-loop device in this embodiment.

Referring to FIG. 4, for example, the first cooling pack unit 21 can be applied in a manner that the first and second cooling packs 211 contact respectively a forearm and a corresponding upper arm of the user 300 while the first pack connecting component 212 is juxtaposed with the elbow of the user 300. By extending one end portion of the connecting band 221 of one of the fastening members 22 through selected ring portions 213 of the outer peripheral section 2111 of a corresponding one of the first and second cooling packs 211, and fastening said one end portion to the opposite end portion of the fastening band 221 through the fastening mechanism 222 of a corresponding one of the fastening members 22, the first and second cooling packs 211 can then be fastened respectively to the forearm and the upper arm of the user 300 without hindering the movement of the forearm relative to the upper arm, thereby resulting in relatively high flexibility during use.

It should be noted that the number of the fastening members 22 may vary in other embodiments of this invention as long as the first and second cooling packs 211 can be properly fastened to the body part of the user 300. Moreover, in other embodiments of this invention, the fastening mechanism 222 of each of the fastening members 22 may be configured as a releasable buckle mechanism or a snap fastener. Furthermore, the cooling pack device 2 of this invention is also capable of cooling food and beverages in the abovementioned manner.

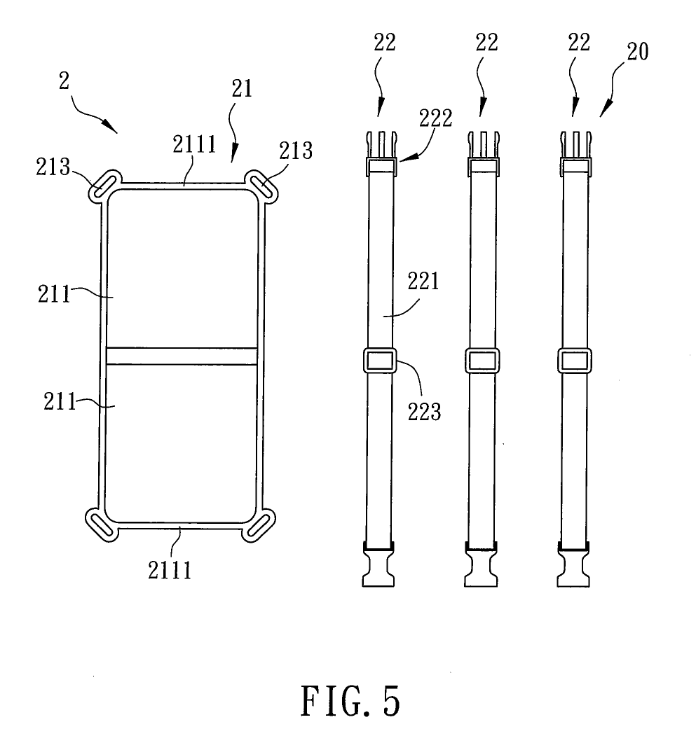

As shown in FIG. 5, the second preferred embodiment of the cooling pack device 2 according to the present invention has a structure similar to that of the first preferred embodiment except for the location of the first ring portions 213 and the configuration of the fastening mechanism 222 of each of the fastening members 22. In this embodiment, each of the first ring portions 213 is formed in the outer peripheral section 2111 of the corresponding one of the first and second cooling packs 211 at a corner of the corresponding one of the first and second cooling packs 211, and the fastening mechanism 222 of each of the fastening members 22 is configured as a releasable buckle mechanism. The second preferred embodiment has the same advantages as those of the first preferred embodiment.

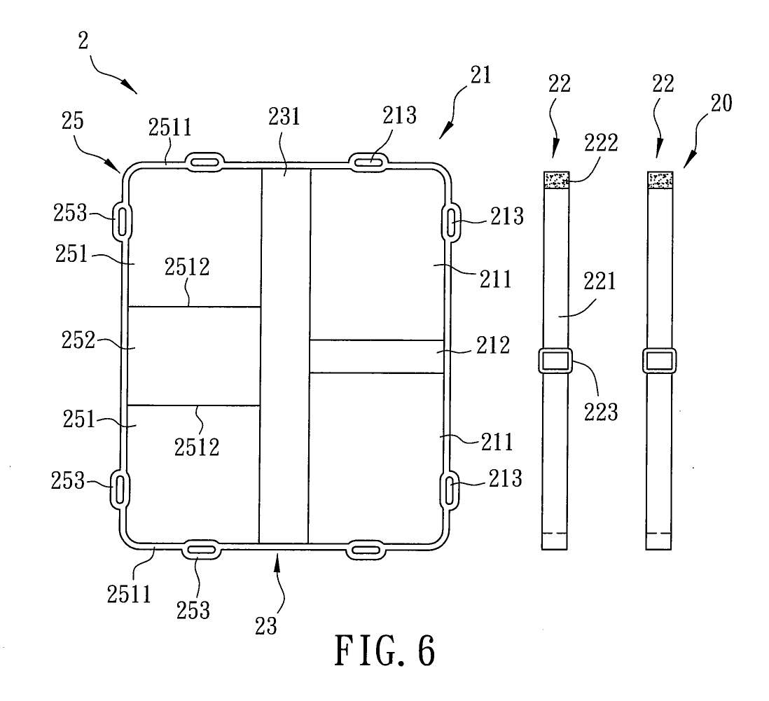

As shown in FIG. 6, the third preferred embodiment of the cooling pack device 2 according to the present invention has a structure similar to the first preferred embodiment, but further comprises a second cooling pack unit 25 and a linking unit 23 interconnecting foldably the first and second cooling pack units 21, 25. The second cooling pack unit 25 includes third and fourth cooling packs 251, each of which has a periphery that has an outer peripheral section 2511 formed with second fastening ring portions 253, and a connecting peripheral section 2512 interconnecting two ends of the outer peripheral section 2511. The second cooling pack unit 25 further includes a second pack connecting component 252 that interconnects foldably the connecting peripheral sections 2512 of the third and fourth cooling packs 251. The linking unit 23 includes a linking member 231 that interconnects foldably the outer peripheral sections 2111 of the first and second cooling packs 211 and the outer peripheral sections 2511 of the third and fourth cooling packs 251.

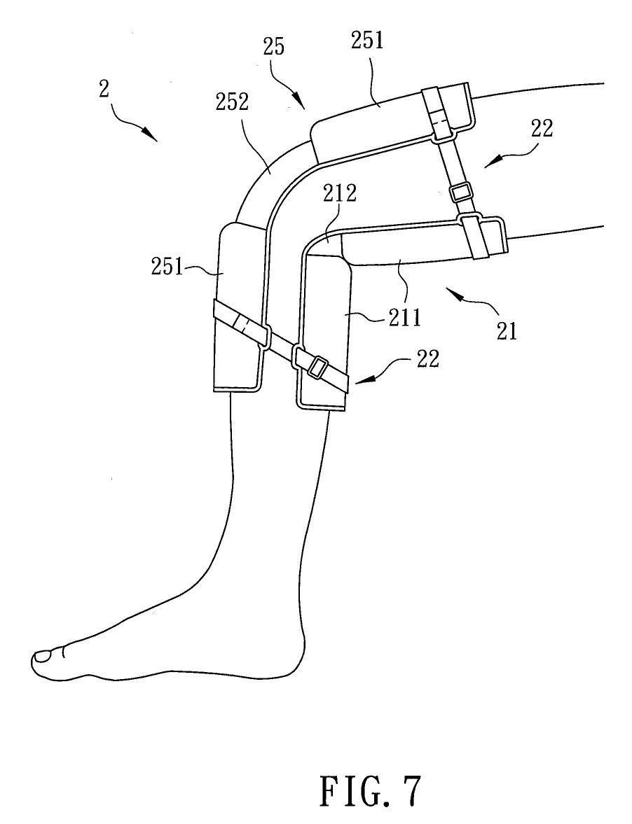

As further shown in FIG. 7, the cooling pack device 2 of this embodiment can be applied to cool a relatively large body part, such as a knee portion. For example, the presence of the linking member 231 (not visible in FIG. 7) enables the cooling pack device 2 of this embodiment to be folded and applied in a manner that the first and second cooling packs 211 of the first cooling pack unit 21 contact respectively lower sides of a thigh and a corresponding calf, and that the third and fourth cooling packs 251 of the second cooling pack unit 25 contact respectively upper sides of the thigh and the corresponding calf. The cooling pack device 2 of this embodiment can then be fastened to the knee portion by the fastening members 22. In use, the first and second pack connecting components 212, 252 are disposed respectively at the lower and upper sides of the knee portion, so that the first and second cooling pack units 21, 25 can be folded in accordance with the bending action of the knee portion, thereby further enhancing the flexibility during use.

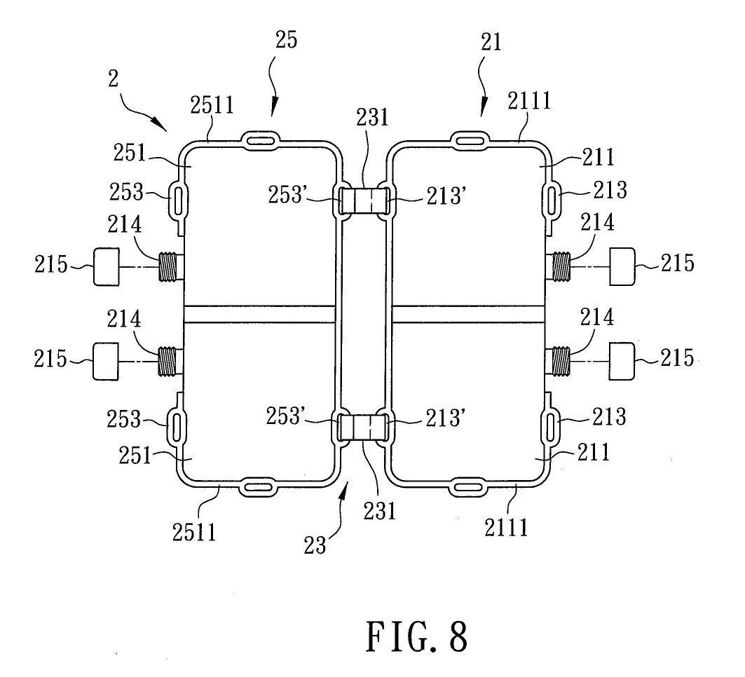

Referring to FIG. 8, the fourth preferred embodiment of the cooling pack device 2 according to the present invention has a structure similar to that of the third preferred embodiment. The main difference between this embodiment and the previous embodiment resides in the following. In this embodiment, the outer peripheral section 2111 of each of the first and second cooling packs 211 is further formed with a first linking ring portion 213′ spaced apart from the first fastening ring portion 213, and the outer peripheral section 2511 of each of the third and fourth cooling packs 251 is further formed with a second linking ring portion 253′ spaced apart from the second fastening ring portion 253.

The linking unit 23 includes a pair of linking members 231. Each of the linking members 231 extends removably through one of the first linking ring portions 213′ of the first and second cooling packs 211 and one of the second linking ring portions 253′ of the third and fourth cooling packs 251. As such, the first and second cooling pack units 21, 25 can be connected to each other through the linking members 231 or be separated from each other by removing the linking members 231, so that the size of the cooling pack device 2 can be flexibly configured.

Each of the first, second, third and fourth cooling packs 211, 251 has an enclosure body formed with an opening 214. The first cooling pack unit 21 further includes a pair of closure members 215 configured as caps and disposed for blocking threadedly, removably and respectively the openings 214 of the enclosure bodies of the first and second cooling packs 211. Similarly, the second cooling pack unit 25 further includes a pair of closure members 215 configured as caps and disposed for blocking threadedly, removably and respectively the openings 214 of the enclosure bodies of the third and fourth cooling packs 25. Before use, each of the first, second, third and fourth cooling packs 211, 251 can either be frozen in a refrigerator, or be filled up with cold water or ice through the openings 214, so that the cooling pack device 2 of this embodiment can be used outdoors. It should be noted that, the combination of the opening 214 and the closure member 215 of each of the first, second, third and fourth cooling packs 211, 251 may be substituted by, for example, a zipper in other embodiments of the invention.

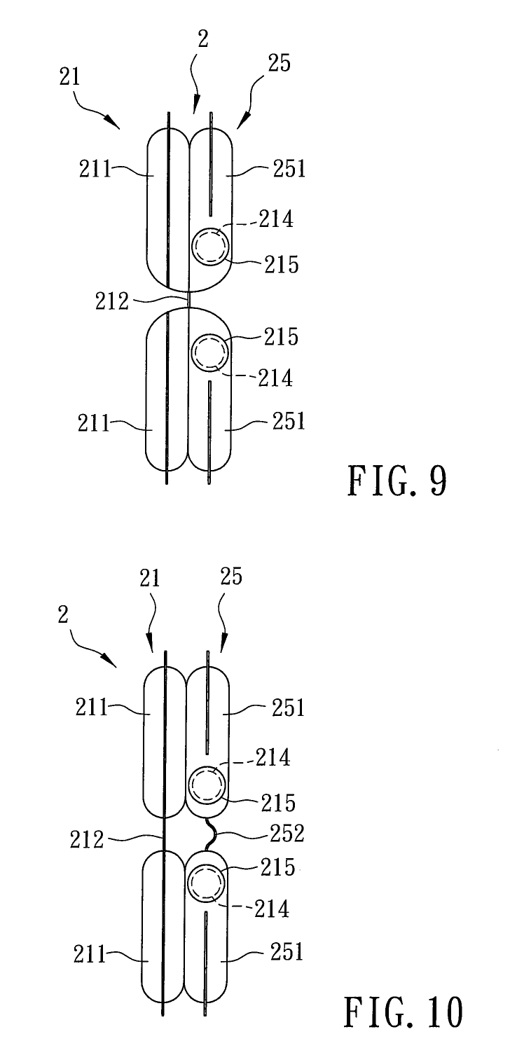

Referring to FIG. 9, the fifth preferred embodiment of the cooling pack device 2 according to the present invention has a structure similar to that of the fourth preferred embodiment. The main difference between this embodiment and the fourth embodiment resides in the following. In this embodiment, the second cooling pack unit 25 includes third and fourth cooling packs 251 that are respectively disposed in heat-conductive juxtaposition with the first and second cooling packs 211. Each of the first and second cooling packs 211 is configured as an airtight enclosure and has a cooling substance (not shown) retained non-removably therein. Each of the third and fourth cooling packs 251 has an enclosure body formed with an opening 214. The second cooling pack unit 25 further includes a pair of closure members 215, each of which is configured as a cap and is disposed for blocking removably a respective one of the openings 214 of the enclosure bodies of the third and fourth cooling packs 251. In use, when temperature of the cooling substances in the first and second cooling packs 211 is not cold enough, cold water or ice can be filled into the third and fourth cooling packs 251 through the corresponding openings 214 so as to prolong the service life of the cooling pack device 2.

As shown in FIG. 10, the sixth preferred embodiment of the cooling pack device 2 according to the present invention has a structure similar to that of the fifth embodiment. The main difference between this embodiment and the previous embodiment resides in that the second cooling pack unit 25 further includes a second pack connecting component 252. The second pack connecting component 252 interconnects foldably the third and fourth cooling packs 251 and is spaced apart from the first pack connecting component 212. The sixth preferred embodiment has the same advantages as those of the first preferred embodiment.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is :

1. A cooling pack device adapted to be applied to an object, said cooling pack device comprising:
a first cooling pack unit including
first and second cooling packs, each of which has a periphery that has an outer peripheral section formed with at least one first fastening ring portion, and a connecting peripheral section interconnecting two ends of said outer peripheral section, and
a first pack connecting component that interconnects foldably said connecting peripheral sections of said first and second cooling packs; and
a fastening unit including at least one fastening member operable for extending through said first fastening ring portions of said outer peripheral sections of said first and second cooling packs so as to fasten said first cooling pack unit on the object.

2. The cooling pack device as claimed in claim 1, wherein each of said first and second cooling packs is configured as an airtight enclosure and has a cooling substance retained non-removably therein.

3. The cooling pack device as claimed in claim 1, wherein:
each of said first and second cooling packs has an enclosure body formed with an opening; and
said first cooling pack unit further includes a pair of closure members disposed for blocking removably and respectively said openings of said enclosure bodies of said first and second cooling packs.

4. The cooling pack device as claimed in claim 3, wherein each of said closure members is configured as a cap.

5. The cooling pack device as claimed in claim 1, wherein said fastening member includes a connecting band having longitudinally opposite end portions, and a fastening mechanism provided on said end portions of said connecting band for connecting releasably said end portions.

6. The cooling pack device as claimed in claim 5, wherein said fastening member further includes an adjusting member provided on said connecting band for adjusting a length of said connecting band.

7. The cooling pack device as claimed in claim 6, wherein said fastening mechanism of said fastening member is configured as one of a hook-and-loop device, a releasable buckle mechanism, and a snap fastener.

8. The cooling pack device as claimed in claim 1, further comprising:
a second cooling pack unit including
third and fourth cooling packs, each of which has a periphery that has an outer peripheral section formed with at least one second fastening ring portion that permits extension of said fastening unit therethrough so as to fasten said second cooling pack unit on the object, and a connecting peripheral section interconnecting two ends of said outer peripheral section, and
a second pack connecting component that interconnects foldably said connecting peripheral sections of said third and fourth cooling packs; and
a linking unit interconnecting foldably at least one of said outer peripheral sections of said first and second cooling packs and at least one of said outer peripheral sections of said third and fourth cooling packs.

9. The cooling pack device as claimed in claim 8, wherein:
said outer peripheral section of each of said first and second cooling packs is further formed with at least one first linking ring portion spaced apart from said at least one first fastening ring portion;
said outer peripheral section of each of said third and fourth cooling packs is further formed with at least one second linking ring portion spaced apart from said at least one second fastening ring portion; and
said linking unit includes a plurality of linking members, each of said linking members extending through said first linking ring portion of one of said first and second cooling packs and said second linking ring portion of one of said third and fourth cooling packs.

10. The cooling pack device as claimed in claim 8, wherein each of said first, second, third and fourth cooling packs is configured as an airtight enclosure and has a cooling substance retained non-removably therein.

11. The cooling pack device as claimed in claim 8, wherein:
each of said first, second, third and fourth cooling packs has an enclosure body formed with an opening;
said first cooling pack unit further includes a pair of closure members disposed for blocking removably and respectively said openings of said enclosure bodies of said first and second cooling packs; and
said second cooling pack unit further includes a pair of closure members disposed for blocking removably and respectively said openings of said enclosure bodies of said third and fourth cooling packs.

12. The cooling pack device as claimed in claim 11, wherein each of said closure members is configured as a cap.

13. The cooling pack device as claimed in claim 8, wherein said fastening member includes a connecting band having longitudinally opposite end portions, and a fastening mechanism provided on said end portions of said connecting band for connecting releasably said end portions.

14. The cooling pack device as claimed in claim 13, wherein said fastening member further includes an adjusting member provided on said connecting band for adjusting a length of said connecting band.

15. The cooling pack device as claimed in claim 14, wherein said fastening mechanism of said fastening member is configured as one of a hook-and-loop device, a releasable buckle mechanism, and a snap fastener.

16. The cooling pack device as claimed in claim 1, further comprising a second cooling pack unit including third and fourth cooling packs that are respectively disposed in heat-conductive juxtaposition with said first and second cooling packs, each of said first and second cooling packs being configured as an airtight enclosure and having a cooling substance retained non-removably therein, each of said third and fourth cooling packs having an enclosure body formed with an opening, said second cooling pack unit further including a pair of closure members that are disposed for blocking removably and respectively said openings of said enclosure bodies of said third and fourth cooling packs.

17. The cooling pack device as claimed in claim 16, wherein said second cooling pack unit further includes a second pack connecting component that interconnects foldably said third and fourth cooling packs and that is spaced apart from said first pack connecting component.

18. The cooling pack device as claimed in claim 16, wherein each of said closure members is configured as a cap.

19. The cooling pack device as claimed in claim 16, wherein said fastening member includes a connecting band having longitudinally opposite end portions, and a fastening mechanism provided on said end portions of said connecting band for connecting releasably said end portions.

20. The cooling pack device as claimed in claim 19, wherein said fastening member further includes an adjusting member provided on said connecting band for adjusting a length of said connecting band.

21. The cooling pack device as claimed in claim 20, wherein said fastening mechanism of said fastening member is configured as one of a hook-and-loop device, a releasable buckle mechanism, and a snap fastener.
